(19) United States
(12) Patent Application Publication
(10) Pub. No.: US 2024/0033055 A1
(43) Pub. Date: Feb. 1, 2024

(54) Dental Device and Method of Use

(71) Applicant: Russell Crockett, Casa Grande, AZ (US)

(72) Inventor: Russell Crockett, Casa Grande, AZ (US)

(21) Appl. No.: 18/247,615

(22) PCT Filed: Oct. 29, 2021
(86) PCT No.: PCT/US2021/057387

§ 371 Date: Mar. 31, 2023

Related U.S. Application Data
(60) Provisional application No. 63/107,205, filed on Oct. 29, 2020.

(51) Int. Cl. A61C 9/00; G06F 30/20

(52) U.S. Cl. CPC A61C 9/0053; A61C 9/008; A61C 9/0093; G06F 30/20

(57) ABSTRACT

A dental device having one or more scan bodies and a frame member is described. Each scan body has a longitudinal axis and a wing region that extends radially outwardly from the longitudinal axis. The scan bodies are attached to dental fasteners in a dental arch and the frame member is attached to the wing regions of the scan bodies to form a physical verification jig. The scan bodies are scanned using an intraoral scanner either before or after attaching the frame member. The scan bodies have a three-dimensional digital image file. CAD software aligns the scanned images to the image files and stiches multiple captures of the dental arch together. A dental device having at least two scan bodies and no frame member is also described. The scan bodies are attached to dental fasteners in a dental arch and the wing regions are positioned so as to converge.

This application is a national stage of, and claims priority to, PCT/US21/57387 filed on Oct. 29, 2021, which claims the benefit of priority to U.S. Provisional Patent Application No. 63/107,205 filed on Oct. 29, 2020, which is incorporated herein by reference in its entirety.

### FIELD OF THE INVENTION

The field of the invention is dental devices, in particular, dental devices for intraoral scanning, tissue retraction, and physical verification jigs.

### BACKGROUND

The background description includes information that may be useful in understanding the present invention. It is not an admission that any of the information provided herein is prior art or relevant to the presently claimed invention, or that any publication specifically or implicitly referenced is prior art.

Intraoral scanners have been available to dentists for decades now. But only recently has it become more common in dental practices around the world. Intraoral scanners are devices that have a small handpiece with cameras and light projectors in it. The handpiece fits in a patient's jaw and projects laser light to accurately measure three-dimensional geometries. Simultaneously, the handpiece also takes multiangle images to stitch a three-dimensional image of the dental anatomy and/or dental implant components that are being scanned. Intraoral scanners are typically accurate enough to replace traditional physical impressions of the patient's jaw that enable dental labs to design and manufacture most dental restorations. However, when it comes to full arch dental implant fixed rehabilitation, the accuracy of intraoral scanners are limited by a multitude of factors.

The limited amount of space in the mouth requires the digital light projection technology of intraoral cameras to capture images from a short-fixed distance of 5 mm to 10 mm. This short-fixed distance limits the field of view of each image that is acquired by the intraoral scanner. To create an accurate digital 3-dimensional record of the patient's full oral anatomy, a series of images and measurements are taken by the intraoral scanner at multiple angles across the dental arch. These images are aligned together with advanced software that recognizes overlapping and well-defined three-dimensional geometric shapes and contours. These overlapping 3-dimensional data are used by the software to accurately stitch the individual images together so it can digitally recreate an accurate 3-dimensional record of the patient's full oral anatomy.

Dental computer aided design (“CAD”) software is the other component of intraoral scanning technologies. Examples include 3Shape Dental Studio and ExoCAD. Each of these software have digital libraries that correspond directly to the geometries of scan bodies that are scanned intraorally so that they can be aligned, identified and positioned relative to the anatomical record in the Dental CAD software. These libraries define the relevant scan data on the scan body that is required during the scanning process to capture a digital record of the three-dimensional position of the corresponding implant components that are fastened to the scan body. There must be sufficient scan data available from the scan to align that data with the library file in the Dental CAD software. In order to determine the three-dimensional positions of the dental implant components from an intraoral scan, a scan body, which is attached to the subject dental implant components, and the adjacent hard jaw anatomy are scanned with the intraoral scanner. The three-dimensional digital file from the intraoral scan of the scan bodies is digitally aligned to the three-dimensional digital representation of the scan body in the dental CAD software. Once these two digital files are aligned, any number of corresponding dental implant components can be imported from the library of that scan body in the dental CAD software. These libraries are typically created by the manufacturer of the implant components for the dental CAD software.

Intraoral scanners work extremely well when there are plenty of hard, unique and well-defined teeth to act as markers for alignment. However, during dental implant fixed or implant retained, full arch rehabilitation, all of the teeth on one arch are removed. All that is left is a lot of gum tissue and three or more dental implants. Without teeth, the intraoral scanner may get confused scanning gum tissue and stop scanning. If the scanner keeps getting confused and stops or stitches two images together that are not actually perfectly aligned, it would be impossible to accurately determine relative implant positions in the dental arch.

To fabricate a prosthesis that will passively fasten to all the dental implants together at once, a clinician must accurately capture the relative three-dimensional positions of each individual dental implant in relation to each of the other dental implants. This is traditionally done with a custom-made physical device called a verification jig. Verification jigs are traditionally made by dental labs using rigid luting materials, dental floss and dental implant impression copings. First a physical impression is made of the patient's mouth in the clinician's office. Then a dental model is poured from the impression with approximate implant positions. Impression copings are fastened to the dental model at each implant site. Dental floss is then strung between the impression copings to act as a lattice structure for the hardened acrylic material. Flowable acrylic material is then used to lute all the impression copings together by flowing it on to the dental floss between the impression copings and around each impression coping. Acrylic material tends to shrink as it hardens. In order to minimize the impact of the error this may produce, the verification jig is then cut between each dental implant position so it can be luted together again in the patient's jaw. By minimizing the amount of luting material that is used when the clinician lutes the verification jig together in the patient's jaw, the error caused by shrinkage will be negligible.

It is extremely difficult to capture an accurate record of the relative three-dimensional positions of dental implants in an edentulous jaw with an intraoral scanner because two of the implants are often going to be too far away from each other to effectively capture the relative positions accurately. It becomes even more difficult when there are four or more dental implants with significant distances between each other. Additionally, blood, saliva and soft gum tissue often confuse intraoral scanners and prevent them from completing the scan.

Intraoral scanners have proven to be highly accurate in single tooth restorations where there are plenty of other teeth. However, scanning a full mouth of dental implants during implant fixed or implant retained, full arch rehabilitation has proven to be a unique challenge that is complicated by both the limitations of the intraoral scanning device and the ever-changing conditions of the oral cavity. Understandably, most clinicians have resorted back to physical impressions with physical verification jigs to capture the exact relative three-dimensional positions of the dental implants at once.

The reason there has been significant research and development to capture dental records like dental implant positions digitally is because digital dentistry is significantly more efficient than analog dentistry. Changes can be made on the fly digitally and require less in person visits with the patient. Digital workflows can reduce the complexity of analog workflows by eliminating steps in the process. Digital workflows reduce variables by introducing better ways to analyze and measure the work that clinicians are doing. Ultimately, digital workflows save time, effort and cost for both the clinicians and their patients. When it comes to full arch dental implant rehabilitations, one of the most complex procedures in dentistry, the potential for significant gains in efficiencies from digital workflows is substantial.

Over the past three to four years extraoral scanners with multiple cameras and wider fields of view have been adapted from other industrial fields to solve this unique problem in dentistry. When you know the fixed distance of multiple cameras capturing overlapping images simultaneously, it would be very simple to extrapolate the exact relative three dimensional positions of each individual dental implant in relation to each other with measurements made by lasers and simple geometry. However, this technology, known as photogrammetry, is extremely expensive and has no other usefulness in dentistry. As a result, they are not widely used. Additionally, the accuracy of photogrammetry is not fully proven by clinical studies and journal articles. There are still too many unknowns and too many variables that have not been considered. This includes manufacturing tolerances of the cameras, calibration devices and scan bodies that are used for these devices.

More recently, dental clinical journal articles have started to describe the use of randomized three-dimensional shapes being placed strategically between dental implants in the mouth in order to act as a bridge for the intraoral scanner as it accurately captures the positions of the dental implants. These articles show a trend toward increased accuracy when these three-dimensional shapes are used. Most of the journal articles describe custom made scanning appliances that are fabricated to fit the patient and are attached to the gum tissue. While the articles do describe some effectiveness of these custom appliances, cost and scale become an issue when considering market viability. Additionally, gum tissue and the presence of saliva and blood are not the best conditions for any kind of appliance that needs to be scanned with absolutely no movement whatsoever. Interestingly, there is no single article published that objectively defines the fit of a prosthesis with any sort of measurement parameters. There is only highly subjective clinical observation by well-trained clinicians. Their opinion of a passively fitting prosthesis is the only criteria by which these studies have been able to measure success by.

JP2018504970A teaches a custom designed and fabricated “trial part,” a jig that screws onto implant sites and provides a scannable structure for improving the accuracy and precision of intraoral scans. The trial part has four posts that serve as scannable structures. The trial part can also have two-dimensional and three-dimensional structure extending between two implants that can be separated and rejoined back together in order to passively fit on to each of the implants as one piece before scanning. JP2018504970A fails to teach a dental device that can be assembled in different configurations to universally fit different sized arches.

U.S. Ser. No. 10/136,969 teaches an orientation appliance that is worn during intraoral scanning and X-ray scanning to provide reference points for full denture restorations. The device is used to gather vertical dimension of occlusion, centric relation, centric occlusion, esthetic parameters, phonetics, and function of the final restoration. The appliance also includes a radiopaque marker. The orientation appliance can be made from one single piece or assembled from separate pieces. U.S. Ser. No. 10/136,969 fails to teach a dental device that universal fits different sized arches. U.S. Ser. No. 10/136,969 also fails to provide details regarding the occlusal surfaces of the orientation appliance or using 3-dimensional geometric shapes to facilitate an intraoral scan.

U.S. Ser. No. 10/363,115 teaches a custom designed and fabricated base frame 400 that can be used as a fiducial marker/scan appliance. It has scan bodies 1102 and other superstructure 1304a that provide reference points for joining sequential scans together. In other configurations, a base frame 200 can be removable from a surgical guide superstructure 400. U.S. Ser. No. 10/363,115 fails to teach a dental device that can be universally sized and fitted to an arch without any prior scans or measurements.

U.S. Ser. No. 10/350,036 teaches a reference frame (“connecting-geometry tool 300”) that is placed inside the arch to provide a cross-arch reference. The frame can be coupled to implants, either directly or indirectly through healing abutments. U.S. Ser. No. 10/350,036 also teaches scan plates 502 that attach to healing abutments 500 and distinct features on the scan plate and abutments are used as reference points. U.S. Ser. No. 10/350,036 also teaches incorporating data from a CT scan. However, U.S. Ser. No. 10/350,036 fails to teach bonding or luting a fully rigid frame to scan plates to rigidly fixate the frame. U.S. Ser. No. 10/350,036 also fails to teach using the rigidly bonded or luted frame and scan body apparatus as a physically verified jig for the purposes of fabricating a prosthesis.

US20180206951 teaches a threaded post with a scannable head that engages directly to the jaw that will be used as an alignment device for multiple scans. US20180206951 also teaches the use of rigid support bars 78 that may be telescoped over implant supported scan bodies 72 to provide a “verification jig” and “pathway for scanning”. However, US20180206951 fails to teach a dental device that comprises one or more wings and a separate base frame that can be coupled to the wings to universal fit the dental device in different sized arches.

WO2016110855 teaches a frame (fiducial element 100) worn in the oral cavity for improving the accuracy of intraoral scans. WO2016110855 also teaches that the frame can have a fitting element that can stretch or be deformed to fit the frame into the oral cavity “to allow simultaneous acquisition of occlusion scan data and fiducial mark scan data.” U.S. Ser. No. 10/111,714 teaches putting adhesives on the arch to improve accuracy of an intraoral scan. WO2016178212 teaches a marker fixation device 710 with a magnetic sensor 720 and marks 730 for improving the accuracy of intraoral scanning. However, none of these references appears to teach a dental device that has wing members that couple dental fastener in an arch and provide a platform for attaching a frame.

The Nexus iOS system by Osteon (www.nexusios.com) out of Australia recently released an intraoral scanning system that uses scan bodies that are longitudinally shaped and designed to bridge across the arch during intraoral scanning to provide accuracy during the scan. They claim high accuracy as each kit is custom made, laser measured, and serial numbered. By doing this, they can make adjustments to correct errors when aligning the scans. However, this product does not appear to describe the use of a dental device that has wing members that couple dental fasteners in an arch and provide a platform for attaching a frame.

Instarisa out of Clovis, CA, USA (www.instarisa.com) has also introduced their own Golf scan body for intraoral scanning. They, similar to Nexus iOS, have designed a scan body that is meant to be used with an intraoral scanner that helps bridge implants together on an edentulous arch for more accurate scanning. They also use a flowable material called ScanDar that is somewhat rigid and is applied around the scan bodies in order to hold them together and provide an even hard surface to scan on. However, this product does not appear to describe the use of a dental device that has wing members that couple dental fasteners in an arch and provide a platform for attaching a frame. They do claim that the ScanDar material can be used to hold all the scan bodies together as one piece to create a physically verified jig, but they do not account for possible shrinkage of the material during the luting process that will compromise the accuracy of the physical jig.

While various dental devices are known for facilitation of intraoral scanning of dental implant positions on an edentulous arch, there remains a need for a dental device that can be premanufactured in mass quantities and allow for universal adaptation to a number of variables that only custom-made appliances have thus far been able to resolve. These variables include and are not limited to the number of implants that will be placed, the size of the mouth that is being treated, and the unique connections of the dental implant components. By universally adapting to these variables, cost and scale become significantly more viable to serve the increasing numbers of dentists taking on full arch rehabilitation.

Additionally, there is a significant need to simplify the various steps of dental implant fixed full arch rehabilitation procedures. Records acquisition can be simplified with a radiopaque device that can act as an alignment tool between CBCT scans. Intraoral scanning can also be more efficient if a device created a level surface for scanning the dental implant positions on an edentulous arch. Intraoral scanning would also be simplified if there was a device that can be scanned during surgery while simultaneously acting as a retraction device that holds back tissue flaps during the scan would facilitate a much more efficient scan.

Finally, in order to verify the accuracy of digitally scanned data and to cement implant fasteners into dental prosthetics, a verified physical jig would be ideal. A device that can act as a physical jig after facilitating an intraoral scan of the dental implant positions, would significantly save time and effort when fabricating a final prosthesis for the patient. The clinician would essentially benefit from the efficiencies of a digital workflow while also being confident in the accuracy of a tried and true physical verification jig.

Thus, there remains a need for improved dental devices and their methods of use.

### SUMMARY OF THE INVENTION

The inventive subject matter provides apparatus, systems, and methods in which a dental device comprises one or more scan bodies and a frame member. Each scan body has a body region with a longitudinal axis and a wing region extending radially outward from the longitudinal axis. A bottom end of the body region is configured to mate with a dental fastener in a dental arch, such as a dental implant component. In some embodiments, a through-hole passes through the body portion of the scan body. The opening is sized and dimensioned to receive a screw for attaching the scan body to the dental fastener.

Once the scan bodies are attached to the dental implant component in the dental arch, the scan bodies and dental arch are scanned with an intraoral scanner. The captured images are aligned with corresponding three-dimensional digital image files in a Dental CAD software library and stitched together to create a digital record of the dental arch.

The frame member is fastened to the wing regions of the scan bodies using a bonding or luting material. In some embodiments, the frame member comprises a lattice structure that is designed to receive and hold a luting material to improve bond strength. After the frame member is luted to the scan bodies, the dental device is removed from the dental arch and can be used as a physical verification jig. In this manner, the dental device provides a highly accurate physical model of the three-dimensional positions of the dental implants in the patient's mouth.

In yet other embodiments, the frame member has one or more three-dimensional features to provide definition and improve accuracy for intraoral scanning. In this embodiment, the frame member is attached to the scan bodies before scanning the dental arch. The scannable features can include geometric shapes such as a hemisphere, a cube, a cone, a pyramid, a cylinder, a cuboid, honeycomb, and a prism. It is also contemplated that one or more of the three-dimensional features has a known dimension that can be used to calibrate a physical dimension with digital dimensional data from an intraoral scanner.

In some embodiments, the scan bodies are configured to rotatably couple with the dental implant components to allow for adjustment of the orientation of the wing members (e.g., the direction that the length of the wing member extends towards). In such embodiments, the wing regions can be rotated and positioned so as to converge at a location within the central region of the dental arch. The scan bodies can be chosen from a selection of different shapes, sizes, and configurations in order to fit different dental arches sized and/or different types of existing dental implant components. Likewise, it is also contemplated that the size of the frame member can be chosen by selecting from a plurality of frames members having different shapes, sizes, and configurations.

The inventive subject matter also provides apparatus, systems, and methods in which a dental device comprises at least two scan bodies and no frame member. In such embodiments, the scan bodies are sized and dimensioned to converge within at the same location in the center region of the dental arch within 5 mm of one another, more preferably, 3 mm, most preferably 1 mm. In some embodiments, the tips of the wing regions are tapered to allow for greater proximity so that all the tips can be captured in one image with an intraoral scanner. The scan bodies are preferably configured to rotatably couple with the dental implant components.

The inventive subject matter also provides apparatus, systems, and methods in which a dental device is used to retract tissue flap during implant surgery. The method includes the steps of cutting soft tissue of a dental arch to create one or more surgical flaps, placing one or more implants in a bone of the dental arch, coupling the one or more scan bodies to the one or more dental fasteners, bonding or luting the frame member to the one or more wing members in a position that holds the one or more surgical flaps in a retracted position, and scanning the dental arch and the frame member after the frame member is affixed to the one or more scan bodies and while the one or more surgical flaps is retracted. The method can further include the steps of obtaining a preoperative scan, an intraoral scan while the dental device is coupled with the dental arch, and aligning the intraoral scan with the CBCT scan. The method can also include the steps of removing the frame member and one or more wing members from the one or more implants as a single unit, and suturing the one or more surgical flaps. The method can further include the step of using the single unit as a physical verification jig.

In yet other aspects, the method can include the steps of fabricating a restoration using the scan of the dental arch and frame member while the one or more surgical flaps are retracted by the dental device, and fitting and affixing the restoration to the dental arch within an 8 hour period after the one or more surgical flaps is sutured.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is an exploded perspective view of a first embodiment of a dental device and a dental arch.

FIG. 2 is an exploded front view of the dental device and dental arch in FIG. 1.

FIG. 3 is an exploded side view of the dental device and dental arch in FIG. 1.

FIG. 4a is a side view of the scan body in FIG. 1 showing a wing region.

FIG. 4b is a perspective view of the scan body in FIG. 1 showing a wing region.

FIG. 4c is a plan view of the scan body in FIG. 1 showing a wing region.

FIG. 5 is an exploded side view of the implant, abutment, screws, and scan body in FIG. 1.

FIG. 6 is an exploded side view of the implant, screw, and scan body in FIG. 1.

FIG. 7 is an exploded perspective view of a second embodiment of a dental device and a dental arch.

FIG. 8 is an exploded front view of the dental device and dental arch in FIG. 7.

FIG. 9 is a perspective view of the dental device and dental arch in FIG. 7.

FIG. 10 is a front view of the dental device and dental arch in FIG. 7.

FIG. 11 is an elevated plan view of the dental device and dental arch in FIG. 7.

FIG. 12 is a perspective view of the dental device and dental arch in FIG. 7 with a luting material.

FIG. 13 is a front view of the dental device and dental arch in FIG. 12.

FIG. 14 is an elevated plan view of the dental device and dental arch in FIG. 12.

FIG. 15 is a perspective view of the dental device and dental arch in FIG. 7 with the frame removed.

FIG. 16 is a front view of the dental device and dental arch in FIG. 15.

FIG. 17 is an elevated plan view of the dental device and dental arch in FIG. 15.

FIG. 18 is a perspective view of the dental device and dental arch in FIG. 15 with a luting material.

FIG. 19 is a front view of the dental device and dental arch in FIG. 18.

FIG. 20 is an elevated plan view of the dental device and dental arch in FIG. 18.

FIG. 21 is an exploded perspective view of the implant, screws, and scan body in FIG. 7.

FIG. 22 is an exploded side view of the implant, screws, and scan body in FIG. 7.

FIG. 23a is a side view of the scan body in FIG. 21.

FIG. 23b is a perspective view of the scan body in FIG. 21.

FIG. 23c is a plan view of the scan body in FIG. 21.

FIG. 24 is an exploded perspective view of another embodiment of an implant, screws, and scan body.

FIG. 25 is an exploded side view of the implant, screws, and scan body in FIG. 24.

FIG. 26a is a side view of the scan body in FIG. 24.

FIG. 26b is a perspective view of the scan body in FIG. 24.

FIG. 26c is a plan view of the scan body in FIG. 24.

FIG. 27 is an exploded perspective view of a third embodiment of a dental device and a dental arch.

FIG. 28 is an exploded front view of the dental device and dental arch in FIG. 27.

FIG. 29 is an elevated plan view of the dental device and dental arch in FIG. 27.

FIG. 30 is an exploded side view of the implant, abutment, scan bodies, and screws in FIG. 27.

FIG. 31 is an exploded perspective view of the implant, abutment, scan body, and screw in FIG. 27.

FIG. 32 is an exploded side view of the implant, abutment, scan body, and screw in FIG. 27.

FIG. 33 is an exploded perspective view of another embodiment of an implant, scan body, and screw.

FIG. 34 is an exploded side view the implant, scan body, and screw in FIG. 33.

FIG. 35a is a side view of the scan body in FIG. 33.

FIG. 35b is a perspective view of the scan body in FIG. 33.

FIG. 35c is a plan view of the scan body in FIG. 33.

FIG. 36a is a side view of another embodiment of a scan body.

FIG. 36b is a perspective view of the scan body in FIG. 36a.

FIG. 36c is a plan view of the scan body in FIG. 36a.

FIG. 37 is a perspective view of a fourth embodiment of a dental device.

FIG. 38 is an elevated plan view of the dental device in FIG. 37.

### DETAILED DESCRIPTION OF THE INVENTION

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

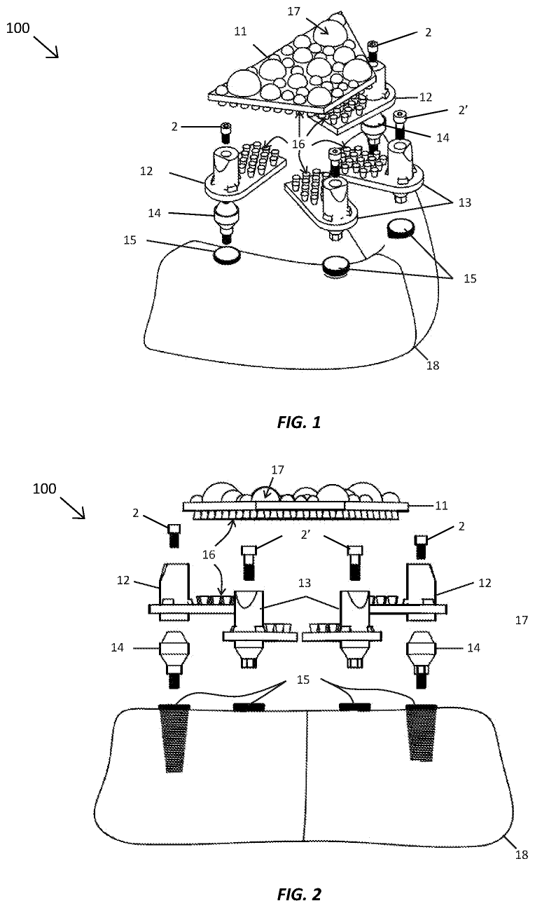

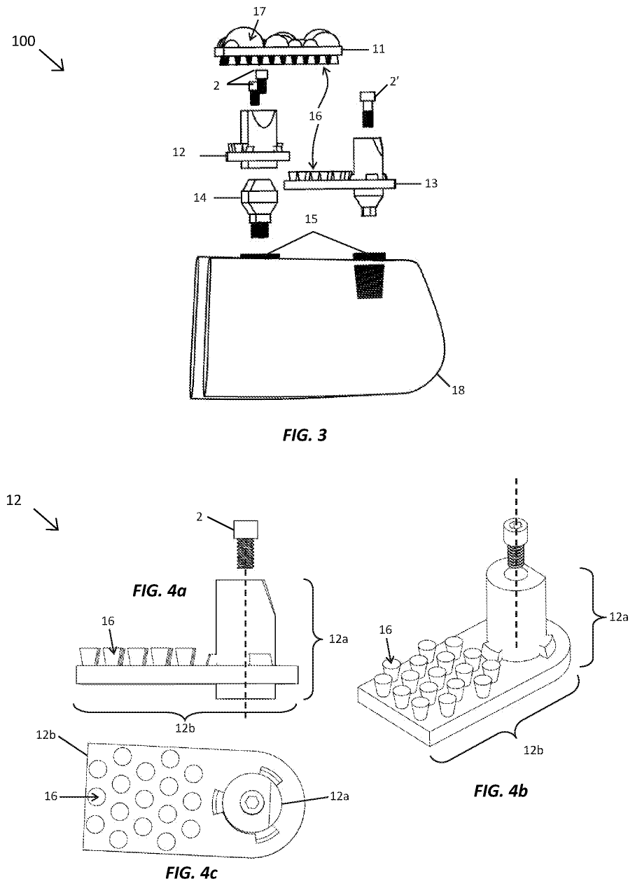

FIG. 1 shows a perspective exploded view of a dental device 100. FIG. 2 shows a front exploded view of dental device 100. FIG. 3 shows a side exploded view of dental device 100. Dental device 100 comprises a frame member 11. Frame member 11 can be milled on a milling machine out of any rigid and strong metal or plastic material. These materials can include titanium, stainless steel, aluminum, PEEK, or PMMA. Frame members can also be 3D printed in resin that is rigid and strong. The gold standard for the ideal 3D printing material would be a dental grade temporary crown material that is designed for 3D printing.

Frame member 11 is coupled with two scan bodies 12 and two scan bodies 13. These scan bodies are manufactured with either milling or 3D printing similarly to the frames above. Manufacturing tolerances for scan bodies are typically highly precise, and currently, milling accuracy is slightly better than the highest quality 3D printing methodologies. Metals like titanium, aluminum or stainless steel can all be used for milling these scan bodies. It is also possible to mill the scan bodies out of plastic materials like PEEK or PMMA. 3D printing, on the other hand, allows for more intricate geometries and undercuts that milling is not able replicate. These intricate geometries and undercuts facilitate luting frame member 11 to scan bodies 12, 13. The most accurate 3D printers available today are the polyjet printers made by Stratasys. These printers are accurate because they can print at a resolution as small as 14 microns. Stratasys also makes the materials for their polyjet printers. Their standard material, Vero, is already excellent for this application due to its high strength and accurate dimensional properties. However, they also make even higher strength dentistry specific materials, like VeroDentPlus Med690. As these materials continue to improve and the 3D printing technology continues to become more accurate, 3D printing may become the manufacturing method of choice for these scan bodies.

Scan bodies 12 are attached to abutments 14 via screws 2. Abutments 14 are attached to implants 15 in dental arch 18. Scan bodies 13 have an abutment portion and a screw 2′ that attaches directly to implants 15.

There are hundreds of dental implant companies on the market around the world. Each make their own screws, abutments and implants. The most well-known dental implant companies include Nobel Biocare, Straumann, Dentsply Implants and Biohorizons. While the dental implants of each of these companies have their own nuances, they all essentially work the same way and have similarly made parts that function the same way. This is even more true with full arch dental implant fixed rehabilitation. The abutment that is generally used for this procedure is the multiunit abutment. Different companies have different names for them, but due to the fact that Nobel Biocare pioneered this procedure, their multiunit abutment was copied by the other dental implant companies. That means that the multiunit abutment across all these different companies is largely very similar, and the parts made to mate with these multiunit abutments are often interchangeable.

Dental device 100 is designed to be scanned by an intraoral scanner after it is luted together on dental arch 18. Luting in dentistry is the process by which a flowable material is injected between two dental components and hardens to attach those two components together. Examples of these components can be any two of a prepped tooth, a dental implant abutment, a crown, a bridge, a prosthesis, temp cylinder, a Ti base or any other similar dental component not mentioned here. The flowable material typically comes in two parts. Those two parts can be any combination of a liquid, a powder, a gel or a paste. When those two parts are mixed, the mixture starts to harden. If the hardened material is chemically similar to the dental components, it may also chemically bond to those components as it hardens. The hardening of the material can be accelerated by blue visible light or UV light if the material is manufactured with a chemical photoactivator that is calibrated to react to a certain wavelength of light. Examples of materials used for luting include PMMA, bisacryl, or composite resin. Specific product examples include Unifast by GC, Chairside by Zest Anchors, Temp by GC, or LuxaTemp by DMG. Once the device is luted together, it can be removed from dental arch 18 as one piece to be used as a physical verification jig.

Dental arch 18 can comprise a maxillary jaw arch or mandibular jaw arch of any person of any age and/or size. Dental arch 18 could also comprise an artificial physical model of a person's maxillary or mandibular arch. The model can be made of various gypsum materials or resin materials.

FIGS. 4a-c show side, perspective, and plan views of scan body 12. Scan body 12 comprises a cylindrical body region 12a and a wing region 12b. The cylindrical body region 12a has a through-hole running longitudinally through the cylindrical body region 12a. There is also a slanted notch on top of the cylindrical body region. This slanted notch facilitates the scan by increasing the surface area of the top of the scan body. This allows for better accuracy and faster scanning speeds. It also gives the scan body more unique character to facilitate the alignment of the scanned data to the three-dimensional digital image file in dental CAD software. Wing region 12b has a length that extends radially outward from the longitudinal dimension of scan body region 12a. Wing region 12b has an attachment area comprising a plurality of pegs or protrusions 16 for holding and gripping luting and/or bonding material. As luting material flows around the pegs, and the luting material hardens around those pegs, the luting material becomes irreversibly attached to the wing region 12a under the undercuts of the pegs. This allows for a stable connection between the wing region 12a and the frame member 11.

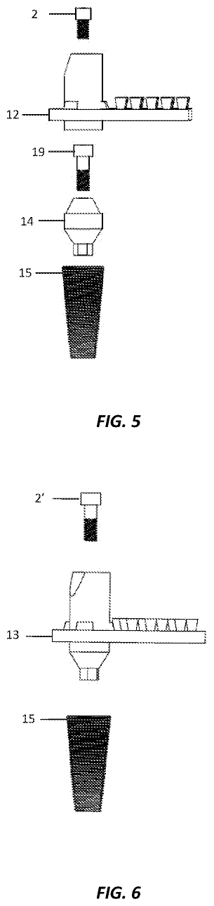

FIG. 5 shows a side exploded view of scan body 12, which is fastened to abutment 14 via screw 2. Abutment 14 is attached to implant 15 via screw 19. Once scan body 12 is placed over abutment 14, it can be rotated to adjust the direction in which wing region 12b extends. After the orientation of scan body 12 is chosen, screw 2 is used to lock scan body 12 in its rotational position.

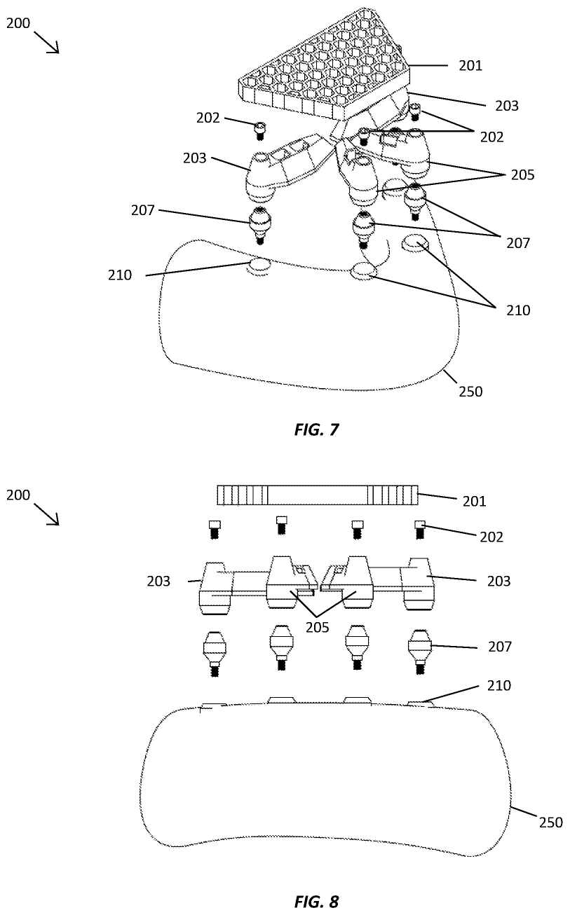

FIG. 6 shows a side exploded view of scan body 13. Scan body 13 is similar to scan body 12 except that the bottom end of scan body 13 is configured to mate directly with implant 15 via screw 2′ without an abutment 14. Scan body 13 shows an embodiment that has a hex that would engage the internal anti-rotational features of implant 15 but it is also contemplated that scan body 13 may not have a hex that engages the internal anti-rotational features of implant 15. In this contemplated embodiment, the scan body can be rotated about the implant freely to adjust the trajectory of the wing region 13b in the dental arch. After the orientation of scan body 13 is chosen, screw 2′ is used to lock the scan body 13 in its rotational position.

FIG. 7 shows an exploded perspective view of a dental device 200 and a dental arch 250. FIG. 8 shows an exploded front view of the dental device 200 and dental arch 250. Dental device 200 comprises a frame 201, two scan bodies 203, two scan bodies 205, and four abutments 207 that fasten with implants 210 in dental arch 250. Scan bodies 203 and 205 have different sizes. Both 203 and 205 have identically sized conical bodies 203a and 205a but differ in size in their wing regions 203b and 205b. wing region 203b is larger and is 19 mm long by 10 mm wide by 5 mm tall. Scan body 203 is generally used in larger spans such as the posterior of the mouth. Wing region 205b is smaller and is 13 mm long by 6.5 mm wide by 5 mm tall. The scan body 205 is generally used in smaller areas of the mouth such as the anterior or on adjacent implants that are close to each other. The actual measurements of scan bodies 203 and 205 are only relevant in that they can be fastened to the abutment 207 without impediment. It is also important that wing regions 203b and 205b are able to converge and either overlap or touch each other at the center of the dental arch. In this case two different sizes of wing regions were contemplated to accomplish these goals. It is also contemplated that one size may be sufficient to accomplish these goals. It is also contemplated that any number of sizes greater than two may be required to accomplish these goals. The conical body of scan bodies 203 and 205 are identically sized. This allows for the layering of digital scan data over time if necessary.

Dental device 200 is designed to be scanned by an intraoral scanner before frame 201 is luted or bonded with scan bodies 203 and scan bodies 205. After scan bodies 203 and 205 are scanned, frame 201 is used to lute the scan bodies 203 and 205 together so the device can be removed as one single piece to act as a physical verification jig. However, it is also contemplated that dental device 200 can be scanned after frame 201 is attached with scan bodies 203 and 205. Frame 201 has a top side, a bottom side and a middle lattice section that has honeycombed shaped through-holes from the top side to the bottom side. Frame 201 is shaped like a trapezoid and has a thickness of approximately 5-10 mm. It is contemplated that frame 201 can be shaped like a triangle, a square, a parallelogram or any other geometric shape that would fit into the patient's jaw and facilitate the luting of the scan bodies together. It is also contemplated that there would be a longitudinal slice of negative space through the middle of the frame that is sandwiched between two honeycomb lattices in the middle lattice section of the frame. This slice of negative space in the middle of the thickness of frame 201 would function as an undercut that luting material would be able to harden around to add rigidity and stability to the physical verification jig that dental device 200 would become. It is also contemplated that any lattice or geometry that could act as a scaffold with a plethora of undercuts to hold luting material would make a suitable frame 201 for dental device 200. It is also contemplated that the size and shape of the frame could easily be adjusted to fit the patient's jaw and position of the wing regions of the scan bodies. The clinician could use any standard dental instrument such as scissors, pliers, electronic handpieces with roughened rotational burs and even their own hands to break off parts of the frame that may be preventing them to lute the frame to the scan bodies effectively.

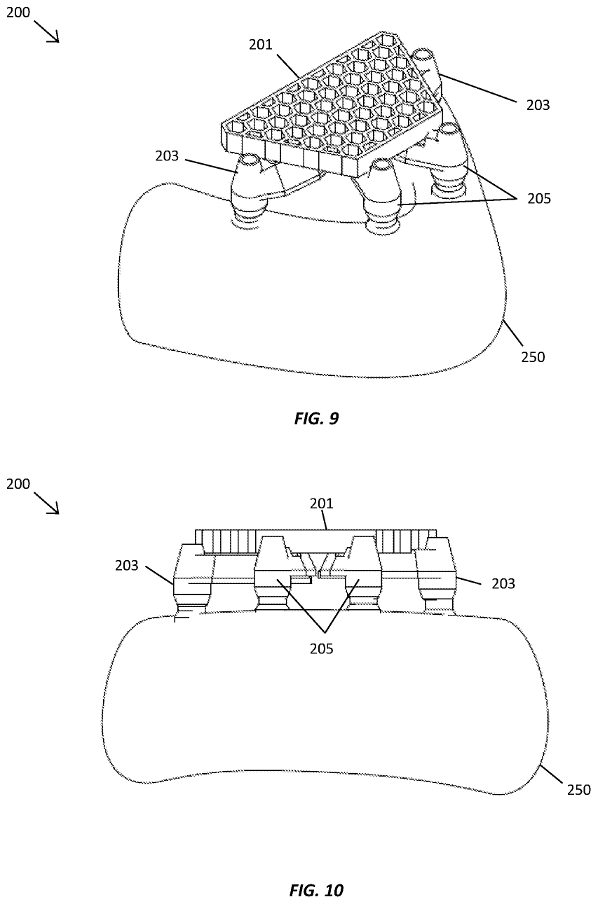

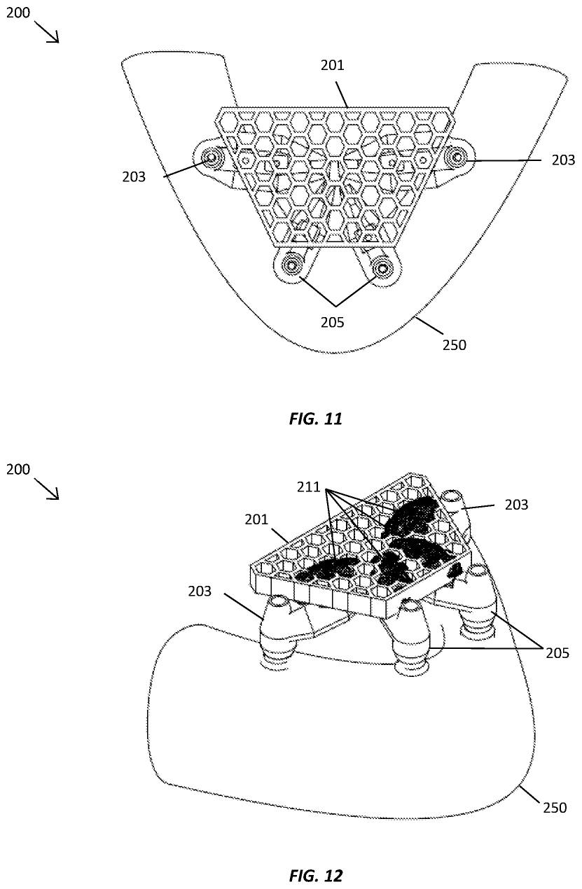

FIG. 9 shows a perspective view of dental device 200 with frame 201 placed on the wing regions of scan bodies 203 and 205. FIG. 10 shows a front view of dental device 200 with frame 201 placed on the wing regions of scan bodies 203 and 205. FIG. 11 shows an elevated plan view of dental device 200 with frame 201 placed on the wing regions of scan bodies 203 and 205.

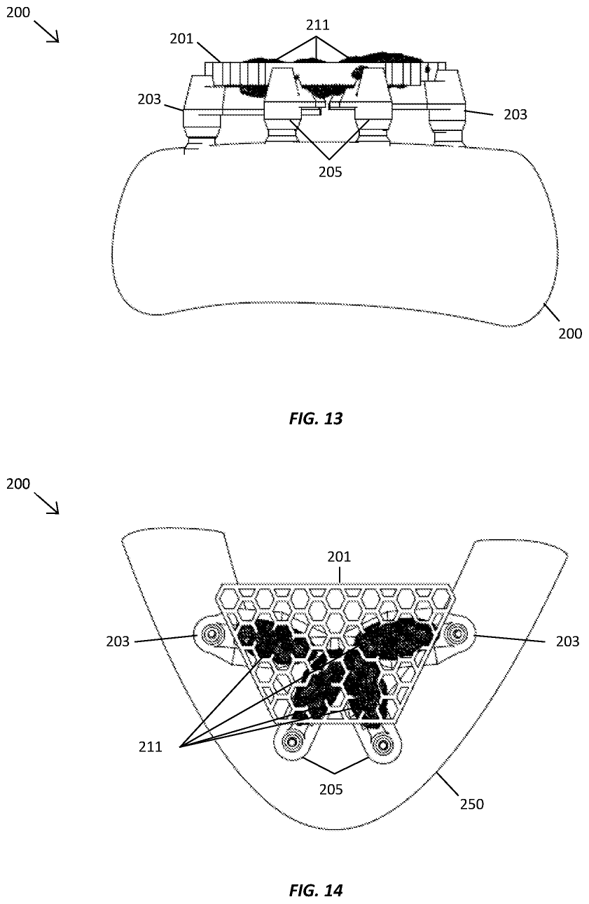

FIG. 12 shows a perspective view of dental device 200 with a luting material 211 between frame 201 and scan bodies 203 and 205. FIG. 13 shows a front view of dental device 200 with a luting material 211 between frame 201 and scan bodies 203 and 205. FIG. 14 shows an elevated plan view of dental device 200 with a luting material 211 between frame 201 and scan bodies 203 and 205.

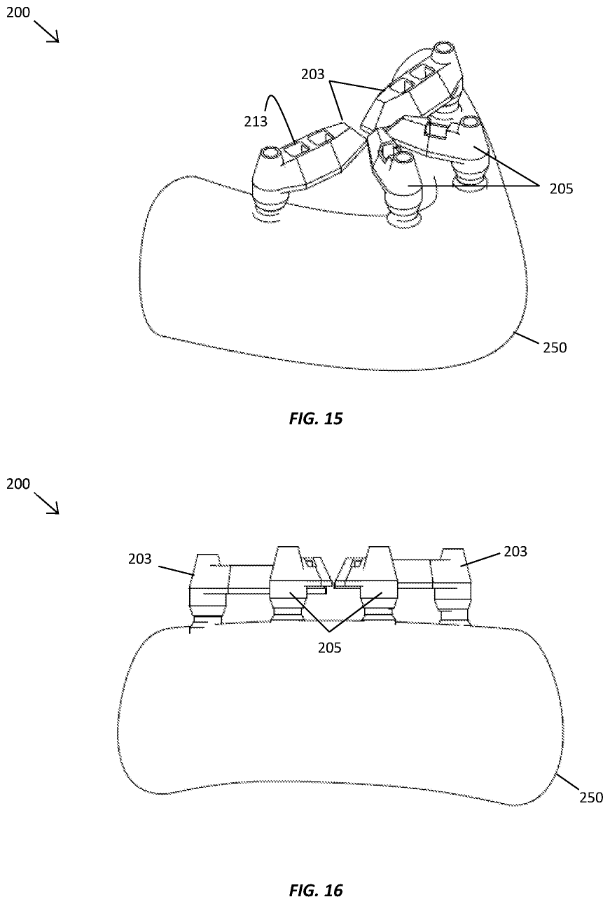

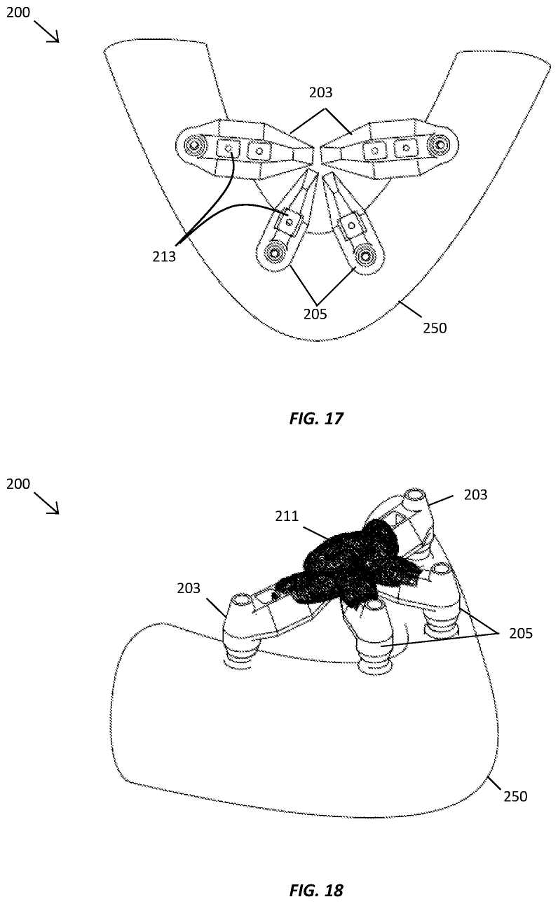

FIG. 15 shows a perspective view of dental device 200 without frame 201. FIG. 16 shows a front view of dental device 200 without frame 201. FIG. 17 shows an elevated plan view of dental device 200 without frame 201. The scan bodies 203 and 25 are positioned so as to converge to a meeting point. The most distal area of wing regions 203b and 205b from the conical bodies 203a and 205a are tapered toward the most distal point so that any number of scan bodies 203 and 205 could nest together to the smallest point possible. When the intraoral scanner is able to capture all of the ends of the scan bodies that are present in one photo frame, it can be expected that the digital record of the three-dimensional positions of the implants is more accurate than if it not all of the wing regions are captured in one frame. The taper of the ends of the wing regions 203b and 205b enables the clinician to fit more wings into a smaller area. The height of each scan body and the rotational position of each scan body are positioned and adjusted so as to come in close proximity with the other scan bodies. In this position, scan bodies 203 and 205 are ready to be scanned and then luted together. In some embodiments, the tips are within 5 mm of contacting each other, more preferably 3 mm, most preferably 1 mm. The proximity of the wing regions creates overlapping data that is scanned in one frame of the intraoral scanner to facilitate more accurate scan data. Additionally, the proximity of the scan bodies to each other facilitates the luting together of the scan bodies to each other. If the scan bodies are close enough together, a frame may or may not be necessary to lute the scan bodies together.

Scan bodies 203 and 205 also have wells 213. These wells 213 give the luting material negative space to lay a strong solid foundation that will not be messy and drip into the patient's mouth. The inside of the well has undercuts that the luting material can flow under to facilitate the attachment of the wing region to a frame. As the luting material fills the inside of well 213 and hardens, the undercuts will prevent the hardened luting material from disengaging from the scan body. The undercuts can either be created at the time of manufacturing or added later by tapping well 213 with a screw tap or cutting the inside of well 213 with a bur. In addition to well 213, any three-dimensional support structure that can be used to hold luting material can also be contemplated. This may include an internal lattice, an external protrusion, cross bars, pegs or even a flat surface that can bond to luting material. It is also contemplated that a vertical structure can protrude from the top of the wing to provide a barrier to prevent luting material from entering the screw hole of the conical body region.

Another function of the contemplated vertical structure could be to facilitate luting the frame to the scan bodies by leveling the surface of wings that are fastened to off-angle dental implants or dental implants placed at different heights. The vertical structure can be adjusted to the same level as an adjacent wing that is at a higher position. When the structures of the wing regions 203b are level, it is much easier to position the frame 201 across all of the wing regions to ensure a rigid and durable structure that will make up the physical verification jig. And yet another function of the vertical structure is that it has additional lattice or three-dimensional geometries that create more scaffolding for the luting material to attach to. Often when implants are placed at different heights and different angles, it is difficult to maintain an even horizontal plane across all wing regions 203b and 205b. A vertical structure that can also attach to the frame regardless of the height of adjacent wing regions allows for more versatility when luting multiple scan bodies 203 and 205 to frame 201. Additionally, it is contemplated that frame 201 could be adjusted with any common dental instrument so that a larger hole made into frame 201 could fit over and around a vertical structure protruding from the wing region 203b or 205b to further stabilize dental device 200 when it is luted together.

The undercuts make sure that once the luting material is hard, it will hold frame 201 with scan bodies 203 and 205.

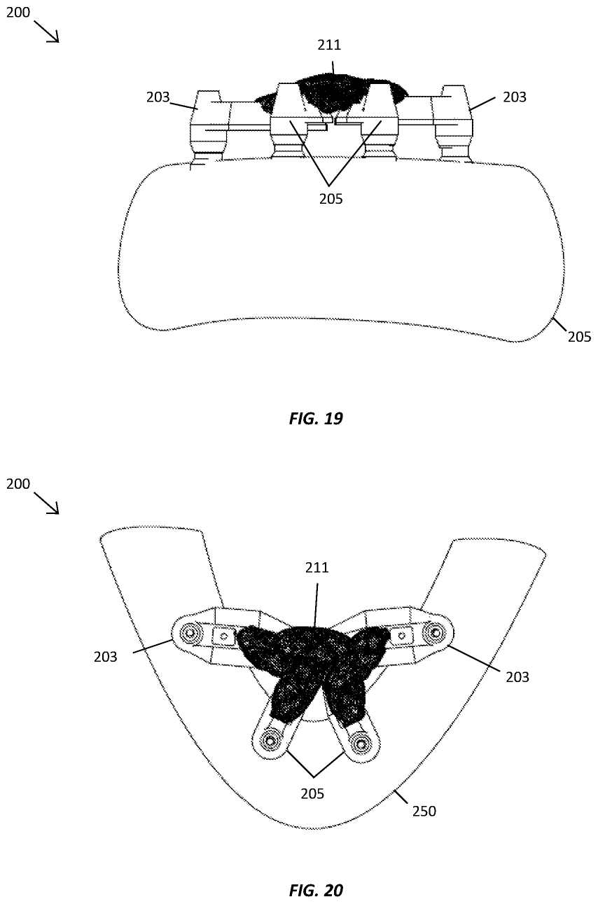

FIG. 18 shows a perspective view of dental device 200 and with luting material 211 holding the scan bodies 203 and 205 together as one single piece without a frame 201. FIG. 19 shows a front view of dental device 200 without frame 201 and with luting material 211. FIG. 20 shows an elevated plan view of dental device 200 without frame 201 and with luting material 211. This method of luting the scan bodies together is only possible when the scan bodies are within 1 mm of each other. Wing regions 203b and 205b are designed specifically to nest as close together as possible at the center of the dental arch.

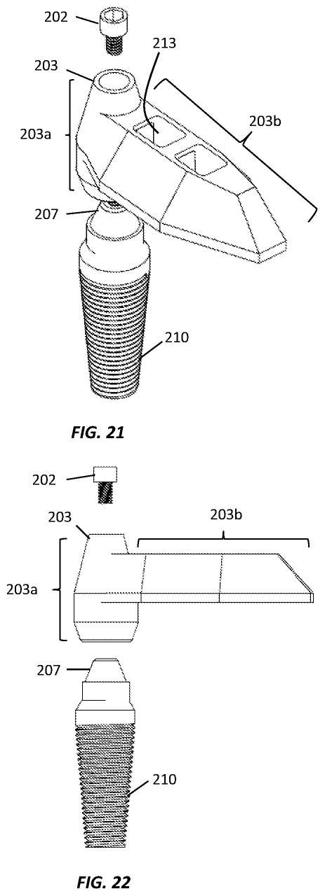

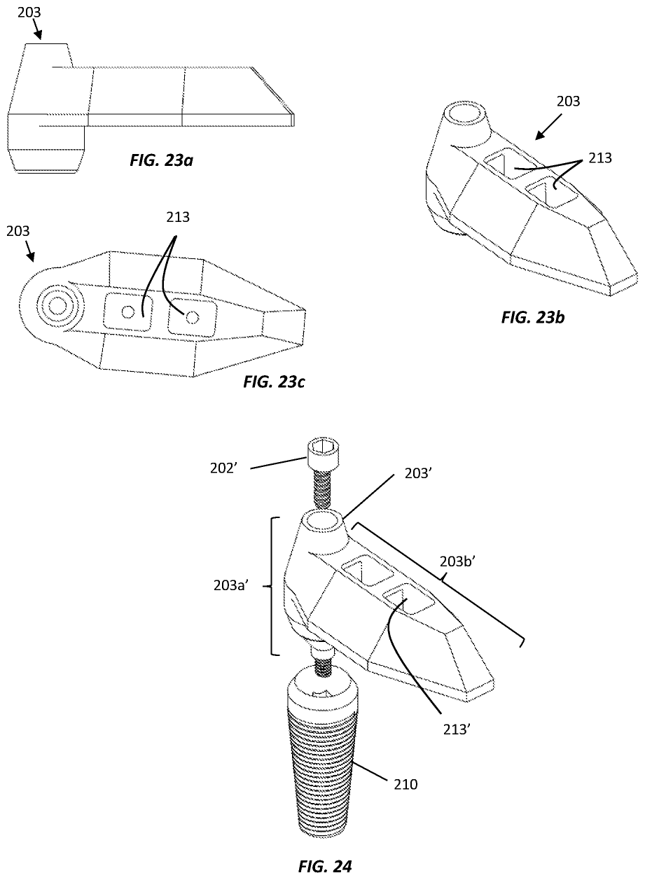

FIG. 21 shows an exploded perspective view of implant 210, screw 202, scan body 203, and abutment 207. FIG. 22 shows an exploded side view of implant 210, screw 202, scan body 203, and abutment 207. FIGS. 23a-c show side, perspective, and plan views of scan body 203. Scan body 203 has a conical body 203a and a wing region 203b extending outwardly from, and perpendicular to, the conical body 203a. The conical body region of scan body 203a has a through-hole running longitudinally through the scan body region. The through-hole allows for a screw to fasten the scan body to a multi-unit abutment. The unique shape and angles of scan body 203 facilitates both the scanning and alignment of scanned data to corresponding digital three-dimensional libraries in dental CAD software. The conical body 203a facilitates intraoral scanning. Intraoral scanners can capture more surface area of the scan body as it moves over the top of the scan body when the scan body is conically shaped. With more surface area to analyze in each frame, higher degrees of scanning speed and accuracy are achievable.

Additionally, other parts, such as the wingless scan body, can also be used during the same procedure and may have the exact shape and dimensions of conical body 203a. Having the same shape and dimensions between different parts allows for the overlapping of digital data whenever these similar parts are scanned throughout the treatment of the patient. This can be useful to capture new or changing information at different time points or milestones of the surgical procedure such as the height of the sutured gum tissue immediately after surgery. Or it can be used to capture and align new information throughout the rest of the treatment as the patient heals and they are ready to receive the final prosthesis. The wing region 203b of scan body 203 has a top surface, two side surfaces and a bottom surface. A cross-section of the wing region would be shaped like a trapezoid, with the top surface that is narrower than the bottom surface. The slanted side panels that taper up to the top surface function similarly to the conical shape of the scan body. The taper allows the intraoral scanner to capture more surface area in each photo frame as it moves over the top of the wing region.

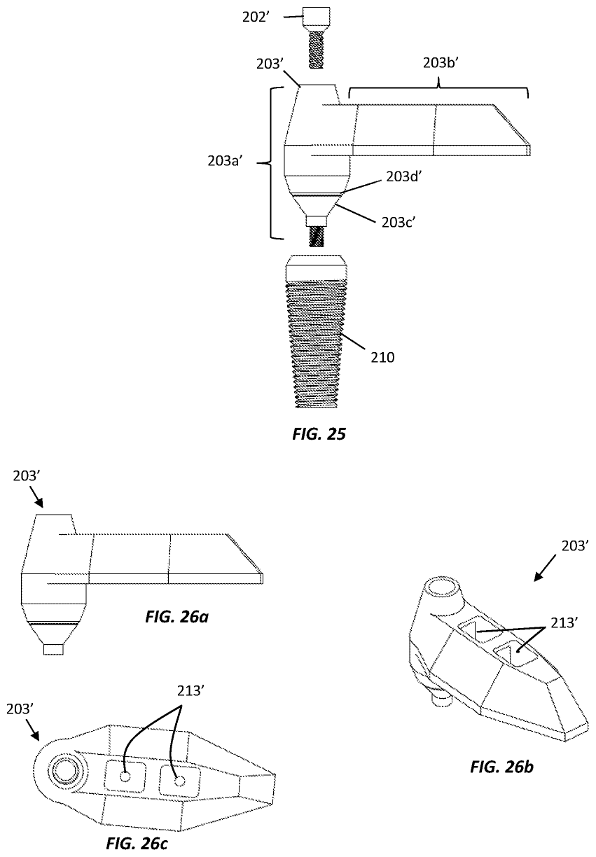

FIG. 24 shows an exploded perspective view of implant 210, screw 202′, and scan body 203′. Scan body 203′ is similar to scan body 203 except for an abutment end 203c′ sized and dimensioned to fit inside implant 210. Screw 202′ is longer than screw 202 and is designed to attached directly to the dental implant 210 with screw threads inside the dental implant. While the dental implant may have a hex or some other anti-rotational feature, abutment end 203c′ does not have a hex that engages the hex inside the dental implant. This would allow the scan body 203′ to rotate about the dental implant until the screw is tightened with enough torque that the scan body will not move. However, it is contemplated that abutment end 203c′ may have a hex that engages with the internal anti-rotational feature of implant 210. It is also contemplated that scan body 203 will have abutment ends 203c that are manufactured to different heights in order to accommodate different tissue heights above the dental implant platform. FIG. 25 shows an exploded side view of implant 210, screw 202′, and scan body 203′. FIGS. 26a-c show side, perspective, and plan views of scan body 203′.

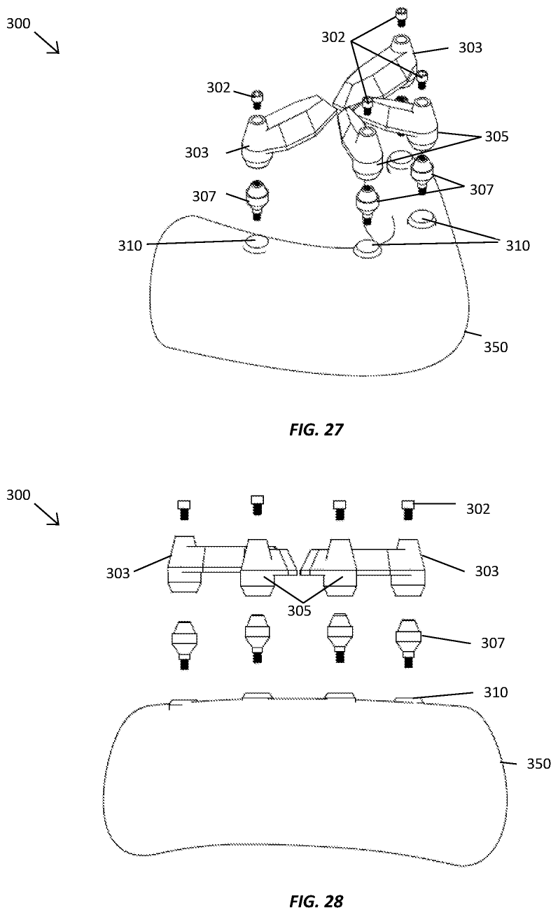

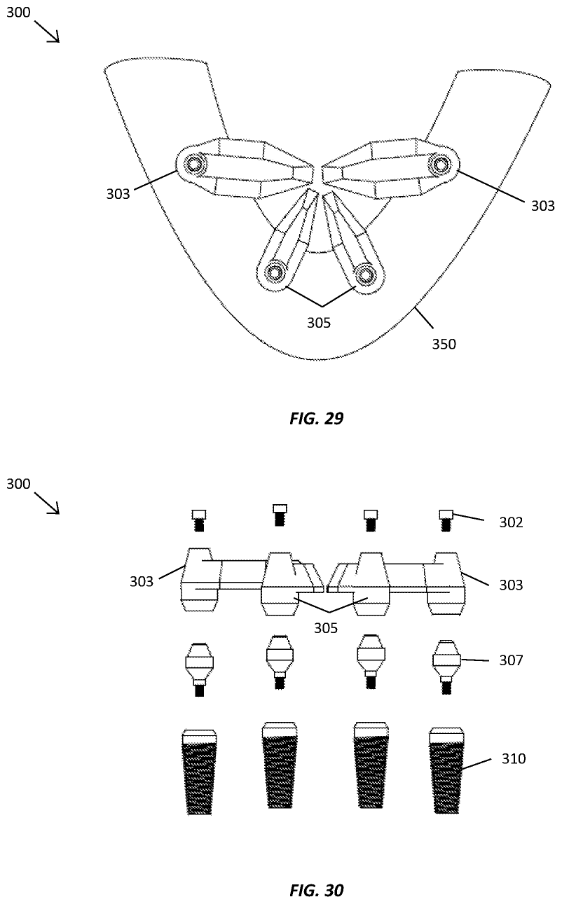

FIG. 27 shows an exploded perspective view of a dental device 300 and a dental arch 350. FIG. 28 shows an exploded front view of dental device 300 and dental arch 350. FIG. 29 shows an elevated plan view of dental device 300 and dental arch 350. Dental device 300 comprises scan bodies 303 and 305 that attach to abutments 307 via screws 302. Abutments 307 attach to implants 310 in dental arch 350. Scan bodies 303 and 305 are similar to scan bodies 203 and 205 except they do not have any wells, openings, holes, channels, undercuts, or grooves for receiving luting material.

Scan bodies 303 and 305 are designed to be scanned only and not luted together. They are also manufactured in a material that allows for them to be reused in subsequent cases after they are sterilized. In some embodiments, scan bodies 303 and 305 comprise a milled titanium that is sand blasted or coated with a matte material that facilitates intraoral scanning. This embodiment has its own corresponding digital library available in the dental CAD software and the scan from these abutments can be used to design prosthetics for full arch dental implant fixed rehabilitation procedures. It is contemplated that these scan bodies 303 and 305 are manufactured with the highest possible manufacturing tolerances currently available at a reasonable cost. Preferably the scan bodies 303 and 305 are manufactured to tolerances within ten microns of variance to the original design specifications. It is contemplated that the digital library will consist of three-dimensional digital image files of the top and side panels of the entire wing region 303b, as well as the top and conical upper portion of the conical body 303a. With more surface area to scan and align, and tighter tolerances during manufacturing, it is contemplated that the accuracy of these scans would be good enough to design and produce prosthetics for full arch dental implant fixed rehabilitation procedures without the need for physical verification. Additionally, scan bodies 303 and 305 must be placed so that all scan bodies 303 and 305 converge at once to as small a point as possible in the center region of the dental arch. Any distance within 5 mm of each wing region tip would be sufficient but 3 mm would be better than 5 mm and any distance within 1 mm would be most ideal to ensure accuracy of the relative three-dimensional data scanned by the intraoral scanner. However, even when the distance is within 1 mm, errors during the scan are impossible to avoid in every instance. If any error is presumed or expected, it is recommended that the clinician make a physical verification jig after scanning to verify the accuracy of the design once the prosthesis is manufactured. This would allow the clinician to make adjustments or corrections before they deliver the prosthesis to the patient's jaw.

FIG. 30 shows an exploded side view of implants 310, abutments 307, scan bodies 303 and 305, and screws 302.

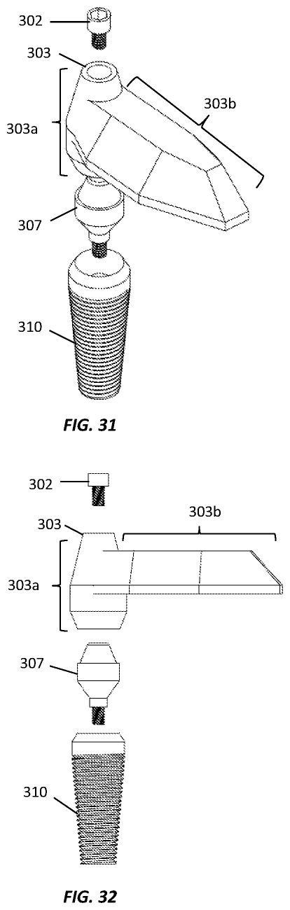

FIG. 31 shows an exploded perspective view of implant 310, abutment 307, scan body 303, and screw 302. FIG. 32 shows an exploded side view of implant 310, abutment 307, scan body 303, and screw 302. Scan body 303 has a conical body portion 303a and a wing region 303b extending outwardly from, and perpendicular to, the conical body portion 303a.

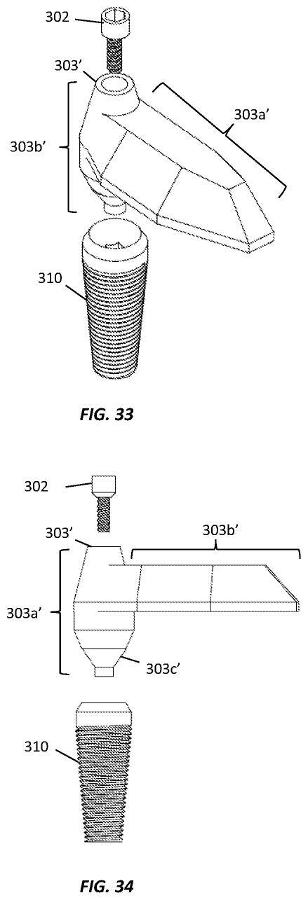

FIG. 33 shows an exploded perspective view of another embodiment of implant 310, screw 302′, and scan body 303′. FIG. 34 shows an exploded side view of implant 310, screw 302′, and scan body 303′. Scan body 303′ is similar to scan body 303 except for an abutment end 303c′ sized and dimensioned to fit inside implant 310. Screw 302′ is longer than screw 302 and is designed to attached directly to the dental implant 310 with screw threads inside the dental implant. While the dental implant may have a hex or some other anti-rotational feature, the abutment end 303c′ will not have a hex that engages the hex inside the dental implant. This would allow the scan body 303′ to rotate about the dental implant until the screw is tightened with enough torque that the scan body will not move. It is also contemplated that scan body 303 will have abutment ends 303c that are manufactured to different heights in order to accommodate different tissue heights above the dental implant platform.

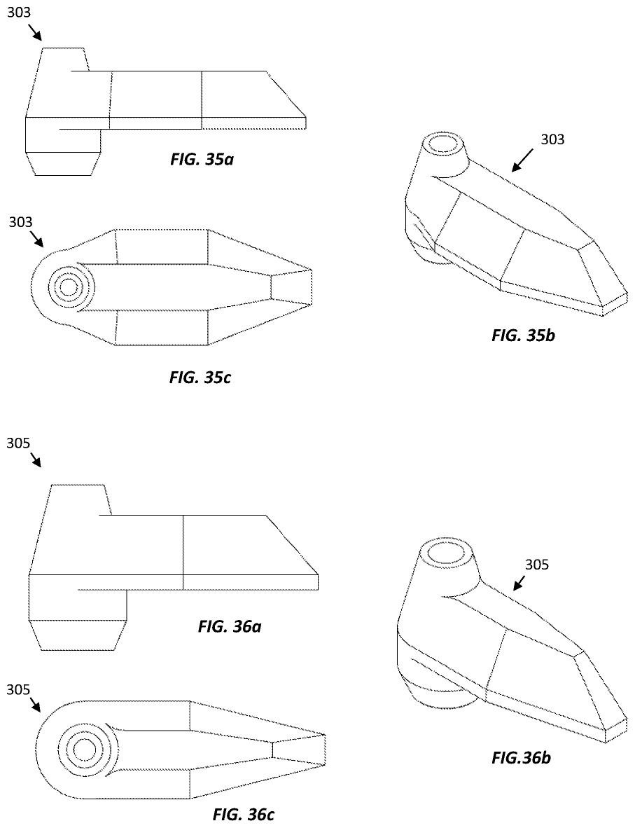

FIGS. 35a-c show side, perspective, plan views of scan body 303.

FIG. 36a-c show side, perspective, plan views of scan body 305.

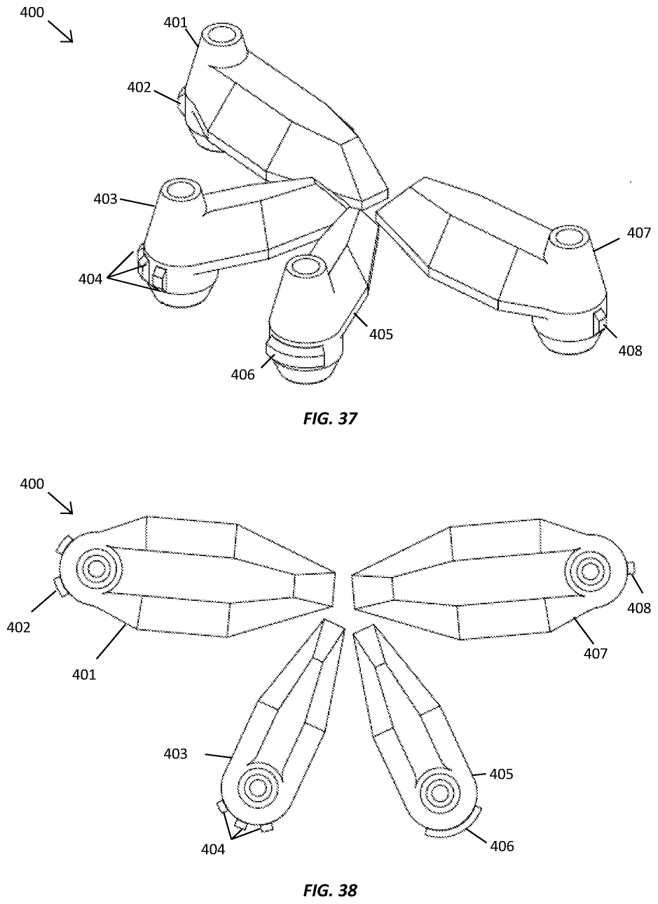

FIG. 37 shows a perspective view of a dental device 400 comprising four scan bodies 401, 403, 405, and 407. Each scan body 401, 403, 405, and 407 has a unique geometric indicator 402, 404, 406, and 408, respectively. The indicators are unique identifiers that may indicate its size, dimension, and other contemplated unique characteristics. Any unique three-dimensional geometry may be used to differentiate one scan body from another. Contemplated geometries include but are not limited to, half-spheres, cones, cylinders, cuboids, or any other polygonal geometry not mentioned here. It is contemplated that these geometries can be of any size and placed in any location on the surface of the scan body as long as they do not impede the positioning, fastening, scanning or luting of the scan body. Additionally, these indicators allow the software of the intraoral scanner to differentiate between each scan body that is currently in the patient's jaw. This would prevent confusing the AI of intraoral scanner so that it does not artificially introduce incorrect or inaccurate scan data during the scan. These indicators are purposely placed below the geometries of scan bodies 401, 403, 405 and 407 that are included in the corresponding digital libraries for these scan bodies in the dental CAD software. Any contemplated indicator of this type would not be used to align the intraoral scan to the three-dimensional digital image file of the scan body geometry. Only the geometries such as the surface of the cone on conical body 401a and the top and side surfaces of wing region 401b would be used to align the two scans. This allows for the convenience of using the same three-dimensional digital image file for scan bodies 401 and 407 and the same three-dimensional digital image file for scan bodies 403 and 405.

FIG. 38 shows an elevated plan view of dental device 400 in FIG. 37.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the amended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification refers to at least one of something selected from the group consisting of A, B, C . . . and N, the text should be interpreted as requiring only one element from the group, not A plus N, or B plus N, etc.

What is claimed is:

1. A dental device comprising:
one or more scan bodies each having a longitudinal axis and a wing region that extends radially outwardly from the longitudinal axis, wherein an end of the one or more scan bodies is configured to mate with a fastener;
a frame member having a top surface and a bottom surface, wherein the bottom surface comprises one or more attachment areas configured to contact an attachment area disposed on the wing region of the one or more scan bodies; and
wherein each of the one or more scan bodies has an associated three-dimensional digital image file available in dental CAD software.

2. The dental device of claim 1, wherein the frame member is sized and dimensioned to fit with a dental arch.

3. The dental device of claim 1, wherein each of the one or more attachment areas of the frame member has one or more protrusions, depressions, holes, undercuts, grooves, or lattice structure.

4. The dental device of claim 1, the attachment area disposed on the wing region of the one or more scan bodies comprises one or more protrusions, depressions, holes, undercuts, grooves, or lattice structure.

5. The dental device of claim 1, wherein scan bodies or the frame member have one or more three-dimensional features with a known dimension that can be used to calibrate a physical dimension with digital dimensional data from an intraoral scanner.

6. The dental device of claim 1, wherein the one or more scan bodies includes a first scan body, a second scan body, a third scan body, a fourth scan body, a fifth scan body, a sixth scan body, a seventh scan body and an eighth scan body.

7. The dental device of claim 1, wherein the frame member and wing members are made of a radiopaque material.

8. A method of using the dental device of claim 1, comprising:
coupling the one or more scan bodies to the one or more dental fasteners;
positioning the wing region of the one or more scan bodies by rotating the scan body about the dental fastener;
scanning the scan bodies with an intraoral scanner;
using dental CAD software to align the scanned three-dimensional image to the three-dimensional digital image file; and
bonding or luting the frame member to the one or more scan bodies by applying an adhesive or hardening material to the attachment areas of the wing regions and the bottom of the frame member and placing the frame member on the one or more wing regions so the frame member is in direct contact with all of the wing regions.

9. The method of claim 8, further comprising the step of CBCT scanning the dental device and the dental arch and aligning the intraoral scan with the CBCT scan.

10. The method of claim 8, further comprising the step of removing the frame member and one or more scan bodies from the fasteners as a single unit and using the single unit as a physical verification jig of the relative fastener positions.

11. The method of claim 8, further comprising the steps of:
selecting the one or more scan bodies from a plurality of scan bodies having different sizes; and
selecting the frame member from a plurality of frame members having different sizes so that the selected frame member fits with a dental arch and rests on each of the one or more wing members.

12. A dental device for scanning a dental arch comprising:
a plurality of scan bodies each having a body region with a longitudinal axis and a wing region that extends radially outwardly from the longitudinal axis;
wherein each body region has a bottom end that is configured to mate with a fastener in the dental arch;
wherein each of the plurality of scan bodies has an associated three-dimensional digital image file available in dental CAD software;
wherein each of the plurality of scan bodies has a shape that facilitates scanning and alignment of scanned data to the associated three-dimensional digital image file;
wherein the wing regions of each of the plurality of scan bodies are sized and dimensioned to converge at a location within a central region of a dental arch and come in close proximity to facilitate luting the scan bodies together.

13. The dental device of claim 12, wherein the wing regions taper longitudinally at the distal end from the screw hole of the scan body.

14. The dental device of claim 12, further comprising a plurality of scan bodies and a frame member each having undercuts on one or more attachment areas configured to couple with each other.

15. A method of using the dental device of claim 12, comprising:
coupling the plurality of scan bodies to the fasteners in the dental arch;
positioning the wing regions of the plurality scan bodies so as to converge within the central region of the dental arch;
scanning the scan bodies with an intraoral scanner;
using dental CAD software to align the scanned three-dimensional image to the three-dimensional digital image file; and
bonding or luting the plurality of scan bodies together.

16. A method of using a dental device comprising a frame member and one or more scan bodies during implant surgery, the method comprising:
cutting soft tissue of a dental arch to create one or more surgical flaps;
placing one or more implants in a bone of the dental arch;
coupling the one or more scan bodies to the one or more implants;
scanning the dental arch and the frame member before or after the frame member is affixed to the one or more scan bodies and while the one or more surgical flaps is retracted; and
bonding or luting the frame member to the one or more scan bodies in a position that holds the one or more surgical flaps in a retracted position.

17. The method of claim 16, wherein the step of scanning comprises obtaining a preoperative scan, an intraoral scan while the dental device is coupled with the dental arch, and aligning the intraoral scan with the CBCT scan.

18. The method of claim 16, further comprising the steps of:
removing the frame member and one or more wing members from the one or more implants as a single unit; and
suturing the one or more surgical flaps.

19. The method of claim 18, further comprising the step of using the single unit as a physical verification jig to verify, and optionally, correct the accuracy of the scanned three-dimensional data.

20. The method of claim 16, wherein the step of bonding or luting the frame member to the one or more wing members comprises leveling the top of the frame member to be at approximately the same height as the top of the scan bodies.

21. The dental device of claim 12, wherein the plurality of scan bodies are sand blasted or coated to facilitate scanning.

22. The dental device of claim 12, wherein each wing region has one or more wells to facilitate luting.
